(19) United States
(12) Patent Application Publication
(10) Pub. No.: US 2008/0010717 A1
(43) Pub. Date: Jan. 17, 2008

(54) Medical Support Assembly Garment

(75) Inventor: Joy J. King, Lake Forest, CA (US)

Correspondence Address: BLAKELY SOKOLOFF TAYLOR & ZAFMAN, 1279 OAKMEAD PARKWAY, SUNNYVALE, CA, 94085-4040, US

(73) Assignee: 2 ASSIST, LLC, Lake Forest, CA (US)

(21) Appl. No.: 11/861,793

(22) Filed: Sep. 26, 2007

Related U.S. Application Data
(63) Continuation of application No. 11/284,525, filed on Nov. 21, 2005, now Pat. No. 7,293,295.

(51) Int. Cl. A41D 1/00; A41D 1/04; A61M 5/00

(52) U.S. Cl. 2/83; 2/102; 604/174

(57) ABSTRACT

A medical support assembly garment dimensionally adapted to receive a torso of a patient. The garment includes a vest portion including a back portion, and a left front portion and a right front portion each extending from opposite ends of the back portion and each comprising an inner surface facing the torso, and at least one pocket attached to the inner surface of at least one of the left and the right portions, wherein the pocket is adapted to support at least one container for storing bodily fluid outputted from the patient.

### FIELD OF THE INVENTION

This invention relates to medical devices. More particularly, the present invention is directed to medical support garments.

### BACKGROUND OF THE INVENTION

Attachment of external drainage devices to a patient's body, such as following a surgery, are common place for a variety of surgical procedures in wide-spread use today.

One area in which these external drainage devices are often attached to patients following a surgery is the operations performed on a patient's breast(s) such as those performed in treatment of breast cancer. Most commonly, breast cancer operations include mastectomy which involve removal of the breast, or lumpectomy which involve removal of lumps from the breast, and often include, or are followed by, some form of breast reconstruction surgery. Following these surgeries, the operating physician often inserts a drainage tube near the operated areas of the patient's breast to reduce accumulations of post-operative fluids, such as blood or lymph, during the convalescence period. The tubes are typically not removed until the drainage output falls to below a predetermined volume per day, such as to below 30 ccs (1 fluid oz).

Generally, these external tubes, often made of rubber or plastic, are secured at one end to patient's body only by sutures. The other end of the tube is typically connected to a reservoir or container in which the fluids are received and collected. As such, movements of the tubes can cause tugging or even tearing of the connecting sutures resulting in pain, discomfort or serious injury to the patient. In addition, the container's weight and swing motion often exerts detrimental force on the tubes to further exacerbate the foregoing problems.

To reduce the adverse impacts of the movement or weight of the container on the tubes, patients are often required to restrain the container from movement. One approach is for the patients to hold the container by hand, which reduces the availability of their hands for every day usage. Other approaches include the securing of the container or the tubes to the patient's clothing or hospital gown, such as via a pin. A shortcoming in the foregoing approaches is that the container and large portions of the tubes remain exposed and prone to impact or entanglements with external objects in the patient's path, such as door knobs, handles or other protruding objects, thus still resulting in tugging or tearing of the connecting sutures. In addition, during activities which require both the removal of a patient's garments and the use of patient's hands, such as showering, the patients are often forced to once again resort to holding the container in their hand(s) which they need for showering and safeguarding against slippage, thus often increasing the chances of shower-related injuries to the patient.

Accordingly, there is a need for providing improved mobility and reduced inconveniences to patients with externally attached drainage devices during both day and night recovery period, and bathing activities.

### SUMMARY OF THE INVENTION

This invention can be regarded as a medical support assembly garment dimensionally adapted to receive a torso of a patient. The garment includes a vest portion including a back portion, and a left front portion and a right front portion each extending from opposite ends of the back portion and each comprising an inner surface facing the torso, and at least one pocket attached to the inner surface of at least one of the left and the right portions, wherein the pocket is adapted to support at least one container for storing bodily fluid outputted from the patient.

This invention can also be regarded as a medical support assembly garment dimensionally adapted to receive a torso of a patient. The garment includes a vest portion including a back portion, and a left front portion and a right front portion each extending from opposite ends of the back portion and each comprising an inner surface facing the torso, and a retaining belt assembly attached to the vest and comprising a first end and a second end, wherein the first and second ends are adapted to circumscribe the torso and to attached to each other to retain the torso within the vest. The garment further includes at least one pocket attached to the inner surface of at least one of the left and the right portions and comprising means for opening and closing of the pocket, wherein the pocket is adapted to support at least one container for storing bodily fluid outputted from the patient, the container comprising a drain tube attached to the container adapted to transfer the bodily fluid outputted from the patient to the container, wherein the vest comprises at least one tab portion adapted to be attached to by the container for restricting a movement of the container, and wherein the pocket comprises the tab portion for restricting a movement of at least one of the drain tube and the container in the pocket.

### BRIEF DESCRIPTION OF THE DRAWINGS

FIG. 1 is a front view of an embodiment of the medical support assembly garment of the present invention.

FIG. 2 is a side sectional view of an embodiment of the present invention.

FIG. 3 is another view of an embodiment of the medical support assembly garment of the present invention.

### DETAILED DESCRIPTION OF THE INVENTION

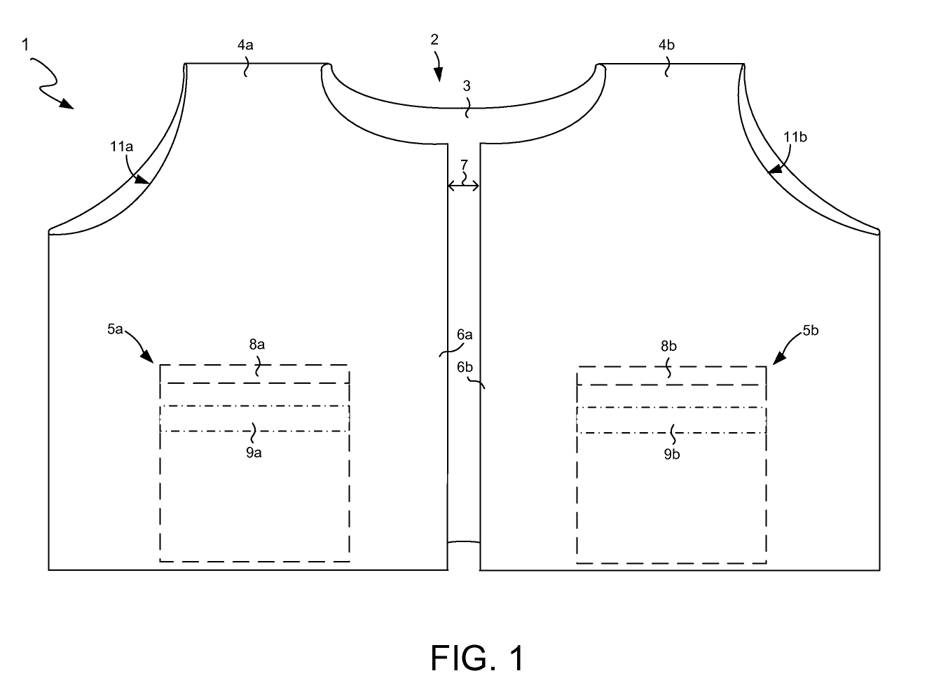

With reference to FIG. 1, an embodiment of the medical support assembly garment 1 of the present invention is shown. The medical support assembly garment 1, such as a post-operative support garment, is dimensionally adapted to receive a torso of a patient (not shown). The medical support assembly garment 1 includes a vest portion 2 including a back portion 3. As shown in FIG. 1, the vest portion 2 further includes a left front portion 4a and a right front portion 4b, each extending from opposite ends of the back portion 3. Each of the left front portion 4a and the right front portion 4b includes an inner surface facing the torso, such as inner surfaces 11a and 11b respectively. Suitably, the medical support assembly garment 1 is of a water-resistant composition, such as a nylon fabric for bathing, or of a cloth composition for non-bathing activities, such as day and night wearing.

The medical support assembly garment 1 further includes at least one pocket, such as pocket 5a or 5b shown in phantom, attached to one or both of the inner surfaces 11a and 11b of the vest 2. Suitably, the medical support assembly garment 1 includes at least two pockets attached to the vest 2, with one pocket, such as pocket 5a, attached to the left front portion 4a and another, such as pocket 5b attached to the right front portion 5b, as shown in FIG. 1. Suitably, pockets 5a or 5b are adjustably or removably attached to the vest, such as via Velcro®. Suitably, each of the pockets 5a and 5b includes means for opening and closing of the pocket, placed on the edge portions 8a and 8b of the pocket 5a and 5b respectively. Suitably, means for opening and closing of the pockets 5a and 5b includes well known mechanism in art for effectuating closures of openings in clothing items such as zippers, complimental set of buttons and hole, strips of hook and loop material, Velcro® etc.

As shown in FIG. 1, the left front portion 4a and the right front portion 4b each include an edge portion, such as 6a and 6b respectively, positioned opposite to each other to define a front opening 7 of the vest 2. Suitably, the vest 2 further comprises means for opening and closing of the front opening 7, placed on the edge portions 6a and 6b. Suitably, means for opening and closing of the front opening 7 includes well known mechanism in art for effectuating closures of openings in clothing items such as zippers, complimental set of buttons and hole, strips of hook and loop material, Velcro® etc.

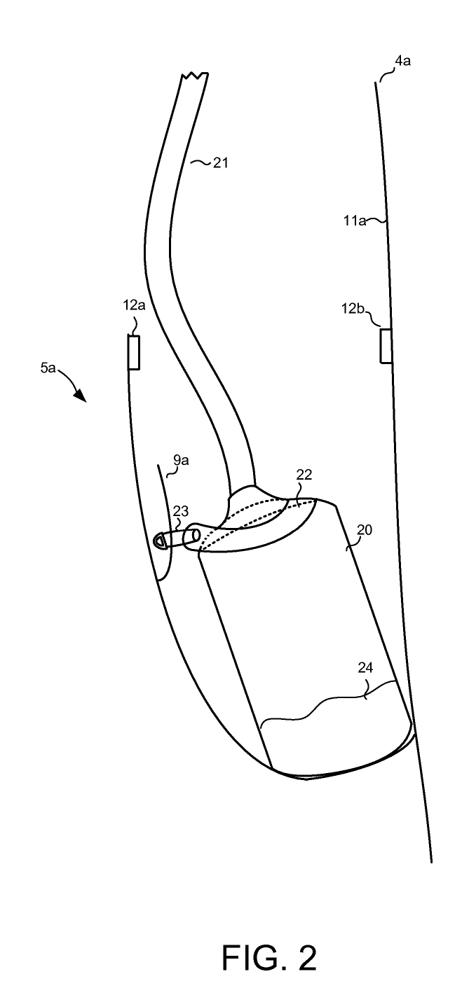

FIG. 2 is a side sectional view of the embodiment of the present invention shown in FIG. 1. For simplicity of illustration, only a cross section in the area of front right portion 4a is shown. As shown in FIG. 2, the pocket 5a is adapted to support at least one container 20 for storing bodily fluid 24 outputted from the patient, such as post-operative fluids, such as blood or lymph, or other fluids. A drain tube 21 is attached to the container 20 and adapted to transfer the bodily fluid 24 outputted from the patient, such as from the patient's breast (not shown) to the container 20. As shown, the pocket 5a comprises the at least one tab portion 9a for restricting the movement of the drain tube 21 or the container 20 in the pocket 5a. Suitably, the drainage bottle 20 is attached to the tab portion 9a via a ribbon 22 attached to the drainage bottle and a pin 23, such as safety pin, attaching the ribbon 22 to the tab portion 9a.

As previously illustrated in FIG. 1, the pocket 5a can be closed along the edge portion 8a, such as by complimental Velcro® strips 12a and 12b as shown in FIG. 2. Suitably, other pockets of vest 2, such as pocket 5b are likewise adapted to support at least one container 20 for storing bodily fluid 24 outputted from the patient and at least one tab portion, such as tab portion 9b in FIG. 1, for restricting the movement of the drain tube 21 or the container 20 in the pocket 5b. Suitably, pocket 5a is used for supporting a container 20 for storing bodily fluid 24 outputted from a patient's right breast and pocket 5b is used for supporting a container 20 for storing bodily fluid 24 outputted from a patient's left breast.

One advantage of the foregoing feature of the present invention over the prior art is that by placing the pockets 5a and 5b (and thus the tubes 21 and container 20) on the inner surface of the front portions 4a and 4b of the vest 2, respectively, outside exposure of the containers 20 and the tubes 21 is reduced, thus reducing the occurrence of impact or their entanglements with external objects in the patient's path, such as door knobs, handles or other protruding objects. In addition, the use of the tab portions 9a and 9b, reduces the need for attaching the container 20 directly to the patient's garment or clothing such by poking holes via the pin 23, thus increasing the longevity and maintaining structural integrity of the patient's garment or clothing. Furthermore, since the medical support assembly garment 1 is suitably of a water-resistant composition, the patient is no longer required to remove the vest 2 during showering, thus freeing the patient's hand and reducing shower related injuries. The medical support assembly garment 1 can also be suitably made of cloth with pockets 5a and 5b to hold the container 20 during sleep periods and during activity periods to hold the container 20 centered under the drain incision for proper placement.

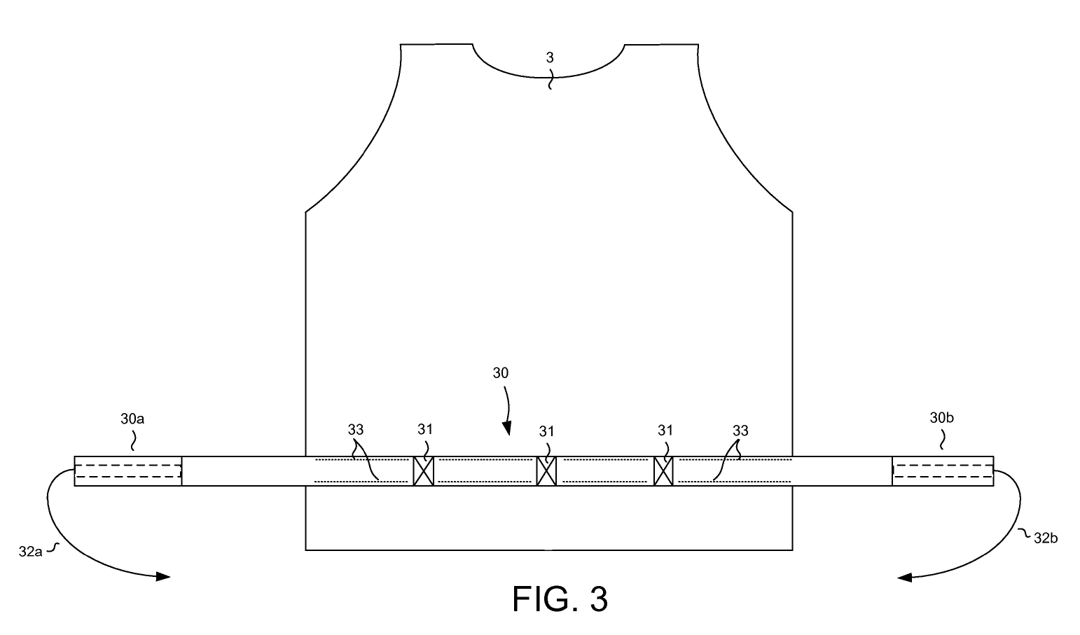

FIG. 3 is a front inside view of the back portion 3 of the medical support assembly garment 1. For ease of illustration, the front left and right portions 4a and 4b are not shown in FIG. 3. In this embodiment of the present invention, the medical support assembly garment 1 further includes a retaining belt assembly 30 attached to the vest 2. As shown in FIG. 3, the belt assembly 30 includes end portions 30a and 30b adapted to circumscribe the patient's torso, such as in the direction shown by arrows 32a and 32b respectively. The end portions 30a and 30b are then adapted to attach to each other to retain the torso within the vest 2, such as via forming a tied knot or via complimental set of buttons and hole, strips of hook and loop material, Velcro® or other well known mechanism in art. Suitably, the belt assembly 30 is retained by or attached to back portion 3 of the vest, as shown in FIG. 3. Suitably, the belt assembly 30 is retained by the back portion 3 via belt loops 31, or attached to the back portion 3 via sewing 33, or via other means of retaining or attaching fabrics well known in the art.

One advantage of the foregoing feature of the present invention over the prior art is that by using the belt assembly 30 to circumscribe and retain the torso within the vest 2, the medical support assembly garment 1 can be more securely and snugly adjusted to fit a patient's body, thus reducing the occurrence of accidental slippage of the vest 2 from the patient's body. In addition, for activities such as bathing and basin while dressing sutures are present, the belt assembly 30 reduces the occurrence of the entry and accumulation of water and other external fluids into the vest 2 and pocket 5a and 5b, thus assisting to keep the inside of the vest 2 relatively dry.

It should be noted that the various features of the foregoing embodiments were discussed separately for clarity of description only and they can be incorporated in whole or in part into a single embodiment of the invention having all or some of these features. It should further be noted that the use of the present invention is not limited to breast operations but can be readily used in conjunction with virtually any medical drainage device that is externally attached to a patients body, such as catheters, and any container adapted to receiving the outputs from the drainage devices.

What is claimed is:

1. A medical support assembly garment dimensionally adapted to receive a torso of a patient, the garment comprising:
a vest portion including a back portion, and a left front portion and a right front portion each extending from opposite ends of the back portion and each comprising an inner surface facing the torso; and
at least one pocket attached to the inner surface of at least one of the left and the right portions, wherein the pocket is adapted to support at least one container for storing bodily fluid outputted from the patient.

2. The garment of claim 1, wherein the container comprises a drain tube attached thereon to and adapted to transfer the bodily fluid outputted from the patient to the container.

3. The garment of claim 2, wherein the vest comprises at least one tab portion adapted to be attached to by the container for restricting a movement of the container.

4. The garment of claim 3, wherein the pocket comprises the at least one tab portion for restricting a movement of at least one of the drain tube and the container in the pocket.

5. The garment of claim 1, wherein the at least one pocket comprises means for opening and closing of the pocket.

6. The garment of claim 1, wherein the left front portion and the right front portion each comprising an edge portion positioned opposite to each other to define a front opening of the vest, wherein the vest further comprises means for opening and closing of the front opening.

7. The garment of claim 1, further comprising:
a retaining belt assembly attached to the vest and comprising a first end portion and a second end portion, wherein the first and second end portions are adapted to circumscribe the torso and to attach to each other to retain the torso within the vest.

8. The garment of claim 7, wherein the belt is attached to back portion of the vest.

9. The garment of claim 1, wherein the garment is of a water-resistant composition.

10. The garment of claim 1, wherein the garment is of a cloth fabric composition.

11. The garment of claim 1, further comprising:
a plurality of pockets attached to the vest, wherein at least one pocket is attached to the left front portion and at least one pocket is attached to the right front portion wherein each pocket is adapted to support at least one container storing fluid outputted from the patient.

12. The garment of claim 1, wherein the at least one pocket is adjustably attached to the vest.

13. The garment of claim 1, wherein the at least one pocket is removably attached to the vest.

14. The garment of claim 1, wherein the garment is a post-operative support garment.

15. A medical support assembly garment dimensionally adapted to receive a torso of a patient, the garment comprising:
a vest portion including a back portion, and a left front portion and a right front portion each extending from opposite ends of the back portion and each comprising an inner surface facing the torso;
a retaining belt assembly attached to the vest and comprising a first end and a second end, wherein the first and second ends are adapted to circumscribe the torso and to attached to each other to retain the torso within the vest; and
at least one pocket attached to the inner surface of at least one of the left and the right portions and comprising means for opening and closing of the pocket,
wherein the pocket is adapted to support at least one container for storing bodily fluid outputted from the patient, the container comprising a drain tube attached to the container adapted to transfer the bodily fluid outputted from the patient to the container,
wherein the vest comprises at least one tab portion adapted to be attached to by the container for restricting a movement of the container, and
wherein the pocket comprises the tab portion for restricting a movement of at least one of the drain tube and the container in the pocket.
